(19) United States
(12) Patent Application Publication
(10) Pub. No.: US 2005/0096563 A1
(43) Pub. Date: May 5, 2005

(54) Oral fluid sampling device and method

(76) Inventor: Greg Liang, Rancho Cucamonga, CA (US)

Correspondence Address: Greg Liang, 7378 OXFORD PLACE, RANCHO CUCAMONGA, CA, 91730, US

(21) Appl. No.: 10/982,238

(22) Filed: Nov. 5, 2004

Related U.S. Application Data
(60) Provisional application No. 60/516,973, filed on Nov. 5, 2003.

(51) Int. Cl. A61B 5/00

(52) U.S. Cl. 600/573; 600/584

(57) ABSTRACT

A device for collecting an expectorated oral fluid specimen for testing comprises a collection chamber having a specimen reservoir space and a headspace, and a vent, and a hollow tube capable of conducting oral fluid from the oral fluid donor's mouth to the specimen reservoir space of the collection chamber. The headspace is for storing oral fluid foams and the vent is for air to escape the chamber when the oral fluid is being collected into the collection chamber. The tube and the collection chamber are either permanently connected or separate with the device having a connection mechanism allowing the connection of the two. The device may further comprise an assay reagent component for detecting an analayte of the oral fluid specimen. The device provides simple means for oral fluid collection and testing, and especially useful in point-of-collection testing of oral fluid specimens.

This application claims priority of U.S. provisional application No. 60/516,973 filed on Nov. 5, 2003.

### FIELD OF THE INVENTION

The present invention is related to devices for the collection of fluid specimens for testing, and is particularly related to collecting and testing oral fluid specimens for the detection of analytes in said specimens.

### BACKGROUND OF THE INVENTION

Most current methods for collecting oral fluid specimens for the detection of analytes remove by a device that is placed the mouth of the donor. Such oral fluid collection methods include absorption, vacuuming, and aspiration.

U. S. Pat. No. 5,393,496 (Seymour, 1995) discloses a saliva-sampling device that utilizes a filter paper type collector to collect a saliva specimen; the specimen is then transferred into a collection container. A sample container is then inserted into the collection container and becomes fluidly coupled thereto, which allows the separation of the collected saliva sample and the saliva collector.

U.S. Pat. No. 6,416,715 (Gambert, et al., 2002) discloses a device for collecting and conveniently releasing saliva from the device. The device has a porous saliva sampler held on a receptacle that is capable of sliding and squeezing the porous saliva sampler and thus releasing saliva from the porous unit.

U.S. Pat. No. 5,922,614 (Cesarczyk, 1999) discloses a device comprising an absorbent member attached to one end of a tube and a sleeve member that snugly fits around the tube. The absorbent member is used for absorbing oral fluid from the mouth. The flexible sleeve is used to squeeze the absorbent member for expressing the oral fluid from the absorbent member.

U.S. Pat. No. 6,440,087 (Sangha, 2002) discloses using an absorbent pad attached to a handle for collecting saliva while avoiding collecting mucosal transudate. A plastic sheet is attached at one side of the absorbent pad to prevent contact of the absorbent pad with the mouth tissue.

U.S. Pat. No. 6,022,326 (Tatum, et al., 2000) teaches an aspiration method for collecting whole saliva from the mouth. An interface section is connected to a wand through a conduit. A vacuum is applied to draw saliva through the interface into a collection chamber.

Oral fluid collection methods using these devices lack efficiency in terms of the time for completing a collection and the volume of oral fluid collected; it normally takes one or several minutes for these methods to collect a small volume, 0.5 milliliter (ml) to 1.5 ml, of oral fluid specimen, which is non-sufficient for some testing methods or when multiple analytes are being tested. Human factors, such as the skill of the operator to locate the oral fluid in the saliva donor's mouth and the degree of cooperation of the specimen donor, affect the collection outcome, which is undesirable for quality control of the collection process and, sometimes, the associated test methods. Furthermore, due to evaporation, vacuuming may result in losses of volatile analytes from the specimen, and absorption may result in analytes being adsorbed onto the absorbent material. Contrarily, oral fluid expectoration method is a donor initiated oral fluid collection method, which provides oral fluid quickly, the specimen is less manipulated and most analytes are well preserved. However, in practice, collecting expectorated oral fluids is sometimes considered intrusive to the specimen donor's privacy and is often not chosen over device-initiated methods. The container for collecting expectorated oral fluid is large for preventing spillage. Such devices take large storage space and are undesirable for transportation.

### SUMMARY OF THE INVENTION

The present new and useful device and its associated methods, as shown in FIGS. 1 to 23, are improved device and methods for collecting oral fluid specimens for testing. When collecting oral fluid with the device of the invention, instead of being spit into a cup container, the oral fluid specimen is expectorated into a straw or tube connected to a specimen collection chamber. Therefore, in comparison with direct expectoration into a cup, the specimen collection device is smaller, the expectoration process is less intrusive, and the chance for spillage of the specimen is reduced. The specimen collection chamber comprises a headspace for containing foams and an air vent capable of allowing air to escape the specimen collection chamber while the oral fluid is being collected. Both the exterior opening of the tube and the vent can be optionally capped off. The collected oral fluid specimen can be stored in the chamber until it is analyzed by an analytical means. Means for removing the collected specimen from the specimen collection chamber and testing the specimen are also disclosed.

The device and methods are especially useful in point-of-collection (POC) testing of oral fluid specimens.

### BRIEF DESCRIPTION OF DRAWINGS

FIG. 1 is a perspective view of a device in accordance with an embodiment of the invention.

FIG. 2 is a cross-section view of the device of FIG. 1.

FIG. 3 is a diagram that depicts how oral fluid specimens are collected using the device of the invention.

FIG. 4 is a perspective view of an oral fluid collection device in accordance with a preferred embodiment of the invention.

FIG. 5 is a perspective view of the translucent collection chamber of the device of FIG. 4 having a specimen reservoir space and a headspace.

FIG. 6 is a perspective view of the tube of the device of FIG. 4 that fits to the open end of the collection chamber of FIG. 5.

FIG. 7 is a perspective view of an optional cap that fits to the opening of the collection chamber of the device of FIG. 4 with the tube detached from the collection chamber.

FIG. 8 is a perspective view of the collection chamber of FIG. 5 caped with the cap of FIG. 7.

FIG. 9 is a perspective view of an oral fluid collection device in accordance with a different preferred embodiment of the invention.

FIG. 10 is a perspective view of the tube of the specimen collection device of FIG. 9.

FIG. 11 is an oral fluid specimen collection device having a translucent specimen collection chamber and a tube having a locking structure that fits to the open end of the collection chamber and prevents the tube from being moved away from the collection chamber in accordance with a preferred embodiment of the invention.

FIG. 12 is a perspective view of the tube of the oral fluid specimen collection device of FIG. 11 having a fin-like structure that locks the tube to the neck area of the collection chamber of the device.

FIG. 13 is a perspective view of an optional cap of the oral fluid specimen collection device of FIG. 11.

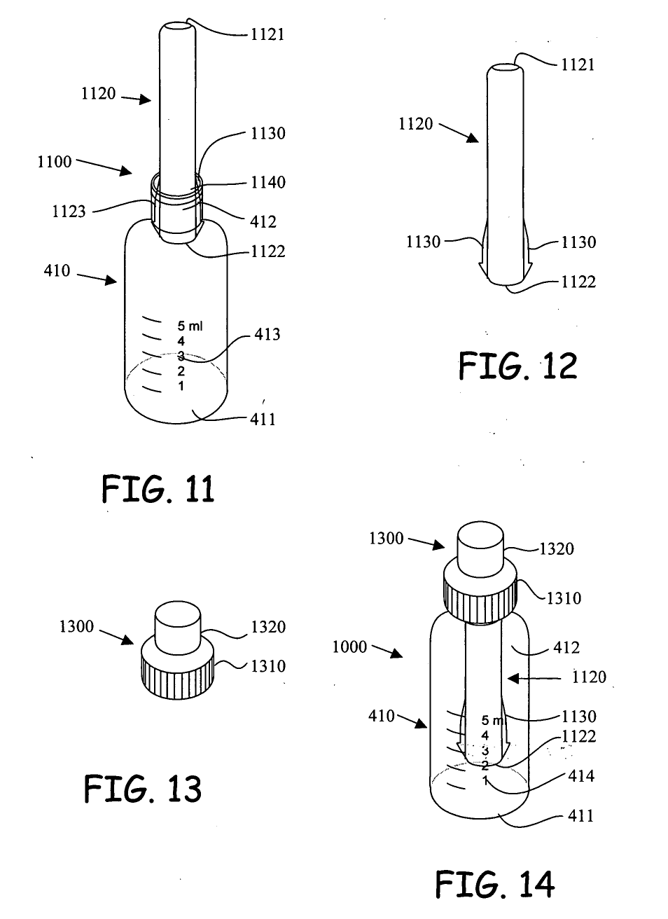

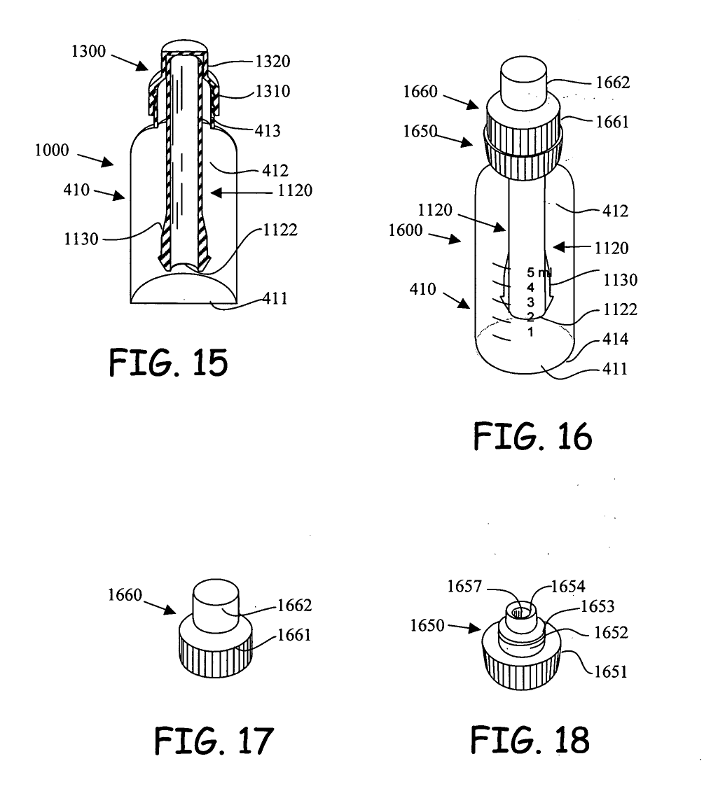

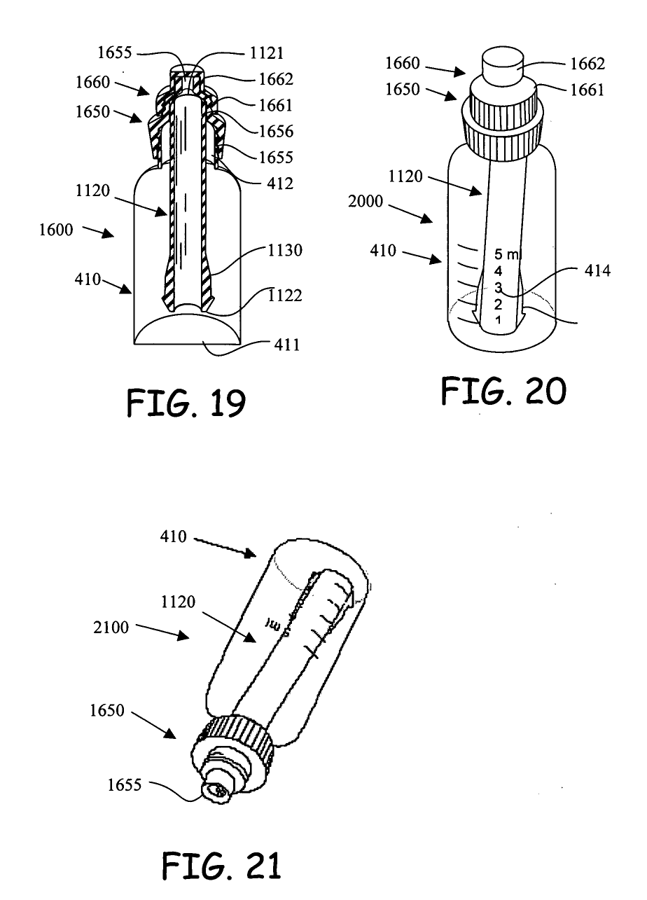

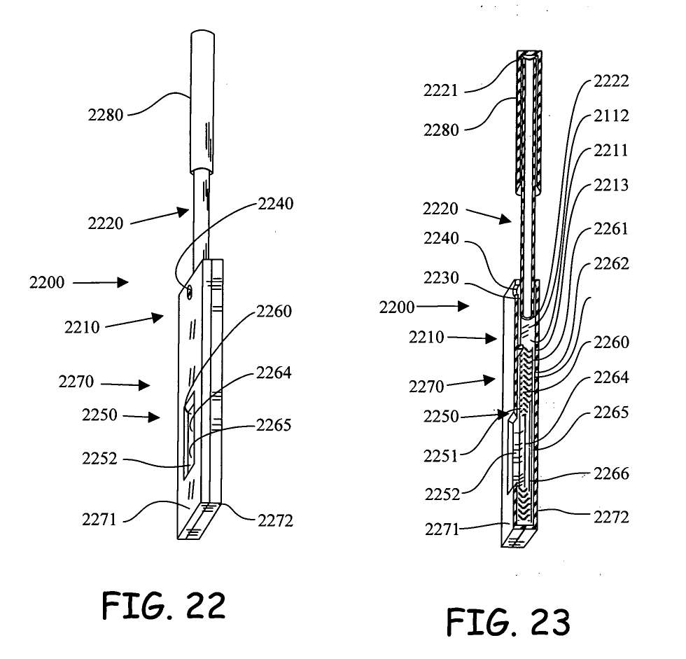

FIG. 10 is a perspective view of the saliva collection device of FIG. 11 caped with an optional cap.

FIG. 15 is sectional view of the saliva collection device of FIG. 10.

FIG. 16 is a perspective view of another saliva collection device that a collection chamber, a tube, and two caps in shipping positions.

FIG. 17 is a perspective view of the top cap of the device of FIG. 16.

FIG. 18 is a perspective view of the drip cap having a through hole of the device of FIG. 16.

FIG. 19 is a sectional view of the oral fluid collection device of FIG. 16.

FIG. 20 is a perspective view of the oral fluid collection device of FIG. 16 after saliva collection.

FIG. 21 is a perspective view of the saliva collection device of FIG. 16 with its top cap off, which illustrates a position in which oral fluid sample is capable of being dripped out of the device for testing.

FIG. 22 is a perspective view of a saliva test device having a tube, an oral fluid collection chamber section and an assay reagent section in accordance with a preferred embodiment of the invention.

FIG. 23 is a sectional view of the device of FIG. 22.

### DETAILED DESCRIPTION OF THE INVENTION

The primary aspect of the invention is a device and method for collecting an oral fluid specimen, i.e., saliva, from a subject. The oral fluid is generally used for medical diagnostic testing but may be used for forensic and other testing, or for banking of the specimen.

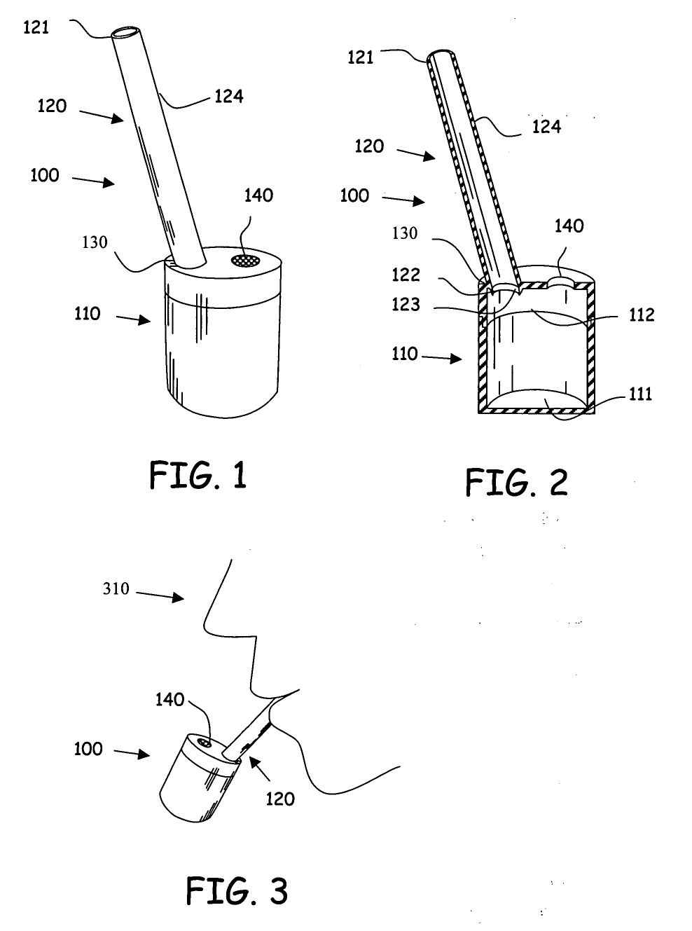

Referring to FIG. 1 in conjunction with FIG. 2, and FIG. 3, the oral fluid collection device (100) comprises a collection chamber (110) having a specimen reservoir space (111) and headspace (112), a tube (120), and a vent (140).

The collection chamber (110) of the oral fluid collection device (100) is a container preferably in the shape of a cylinder having one closed end, although it can be in the shape of a box, a cube, a cone, a barrel, or other shape in some embodiments. The specimen reservoir space (111) is a space within the collection chamber for containing the collected oral fluid specimen when the specimen is being collected. For the fact that oral fluid is usually frothy or foamy, a headspace (112) is necessarily included in the collection chamber (110) for containing foams of oral fluid and for preventing over-flow of the collected oral fluid specimen. The specimen reservoir space (111) and the headspace (112) are adjacent undivided sections of the collection chamber (110). When the oral fluid specimen is being collected in the collection chamber (110), the specimen reservoir space (111) is a space of the collection chamber (110) where the oral fluid is pooled and the headspace (112) is the space of the collection chamber (110) above the specimen reservoir space (111) in altitude, where foams and air reside. The collection chamber (110) is preferably made essentially of a hydrophobic polymeric material such as a plastic or rubber material, or another hydrophobic material, such as glass. The minimum size of the collection chamber (110) varies with the desired volume of oral fluid specimen to be collected, and is generally at least twice the desired volume of the specimen to be collected, i.e., the headspace (112) of the collection chamber should be equal or greater than the specimen reservoir space (111). The preferred volume size of the collection chamber (110) is 2-3 times of the desired volume of the oral fluid specimen to be collected. The desired volume of an oral fluid specimen is usually 2-5 milliliters (ml); therefore, the volume size of the collection chamber is preferably 4-15 ml. The collection chamber (110) is capable of being connected to a tube (120) for conducting oral fluid from the subject's mouth to the collection chamber through an orifice (130) adjacent to the headspace (112). The collection chamber of the device (100) further comprises a vent (140) for allowing air to escape the collection chamber when the oral fluid specimen is being collected.

The tube (120) comprises two ends, a first end (121) capable of being inserted into the oral fluid donor's mouth and a second end (122) capable of being connected with the headspace (112) of the collection chamber (110). The tube (120) is preferred to be tubular in shape with a circular orifice end and in part composed of hydrophobic walls, but may have a flattened; or other shaped orifice end in some embodiments. The shape of the tube (120) is generally cylindrical, and may be either flexible or rigid, depending on application. The tube (120) is preferred to be composed essentially of a hydrophobic polymeric material such as a plastic or rubber material, or another solid material, such as glass. The inner diameter of the tube is preferably 1 millimeter (“mm”) to about 8 mm, and is more preferably 2 mm to 5 mm. The wall thickness should be appropriate for the rigidity and sturdiness of the tube. It was found that the suitable wall thickness is 0.1 mm to 1 mm for a plastic tube (120) and 0.5 mm to 2 mm for a glass or rubber tube (120). The length of the exterior section of the tube, from the first end to the entry of the specimen collection chamber, is preferably 2 cm to 20 cm, and is more preferably 4 cm to 6 cm. Examples of hollow tubes of the device include plastic beverage straws and pipettes. The tube can also be made of separate sections. For example, the tube (120) can be divided into two sections, an interior section (123) that is interior of the collection chamber and an exterior section (124) that is exterior to the collection chamber when the tube is connected with the collection chamber (110). The sections are capable of being in fluid communication.

In a preferred embodiment of the invention, the exterior section of the tube (120), or the entire tube, or the entire device (100) is sterilized, and packaged in sterile packaging containers. Sterile oral fluid collection devices are hygienic, and they prevent microorganisms from contaminating the specimens. The latter may be important in certain types of assay applications. For example, when a microorganism is to be detected in the oral fluid specimen using methods involving tissue culture or polymerase chain reaction (PCR) analysis of the specimen, it may be important to prevent interference of the assay result by microorganisms from the collection device. The method for sterilizing the oral fluid collection device is selected from a group consisting irradiation, ethanol gas sterilization, and steam sterilization.

Unlike in the arrangement of a straw in a beverage container for drawing beverage from the container, where the straw is inserted into the liquid body, the arrangement of the device (100) of the invention is distinctively different by having the second end of the tube disposed in the headspace (112) of the collection chamber (110). One benefit of such arrangement is that bubbling of the specimen due to airflow through the tube into the liquid body of the specimen is avoided. Another benefit of having the second end of the tube disposed away from the specimen reservoir space is the length of the tube can be shorter compared to a tube that extends from the subject's mouth to the bottom of the collection chamber. In some preferred embodiments of the invention, the tube (120) is short enough to be stored inside the collection chamber (110) when the device is not being used for oral fluid collection.

The vent (140) is an opening of the collection chamber wall that connects the headspace (112) of the collection chamber (110) with the atmosphere. The vent (140) allows air in the collection chamber to be displaced by the oral fluid specimen being collected. 8 of 27 Both the tube (120) and the vent (140) can be optionally capped off when the oral fluid specimen is not being collected. A capped off device (100) can be used as a storage container for the collected oral fluid specimen. In a preferred embodiment of the invention, the vent (140) is sealed with a porous membrane that permits airflow and prohibits aqueous solution flow through. Thus, spillage through the vent (140) is prevented when the device (100) contains an oral fluid specimen. Membrane that permits airflow and prohibits aqueous solution flow through can be obtained from varied suppliers, such as Porex Corporation of Fairburn, Ga., USA.

FIG. 3 depicts the method for using the device (100) for collecting an oral fluid specimen is comprised of a subject (310) expectorating oral fluid, i.e., saliva, into a hollow tube (120), with said tube connected to a specimen collection chamber (110), and a vent (140) allowing air to escape the collection chamber (110). The subject is instructed to hold the device (100) in a position such that the specimen reservoir space (111) is substantially below the headspace in altitude such that a liquid is capable of flowing through the tube (120) and the headspace (112) to the specimen reservoir space (111) of the collection chamber (110) by gravity.

In one preferred embodiment of the device of the invention (100), the tube (120) is irremovably connected to the headspace (112) of the collection chamber (110). The tube and the collection chamber are connected by one of a group of methods including molding, gluing, soldering, and welding.

In another preferred embodiment of the device of the invention (100), the tube (120) and the collection chamber (110) are capable of being removably connected through a connection mechanism.

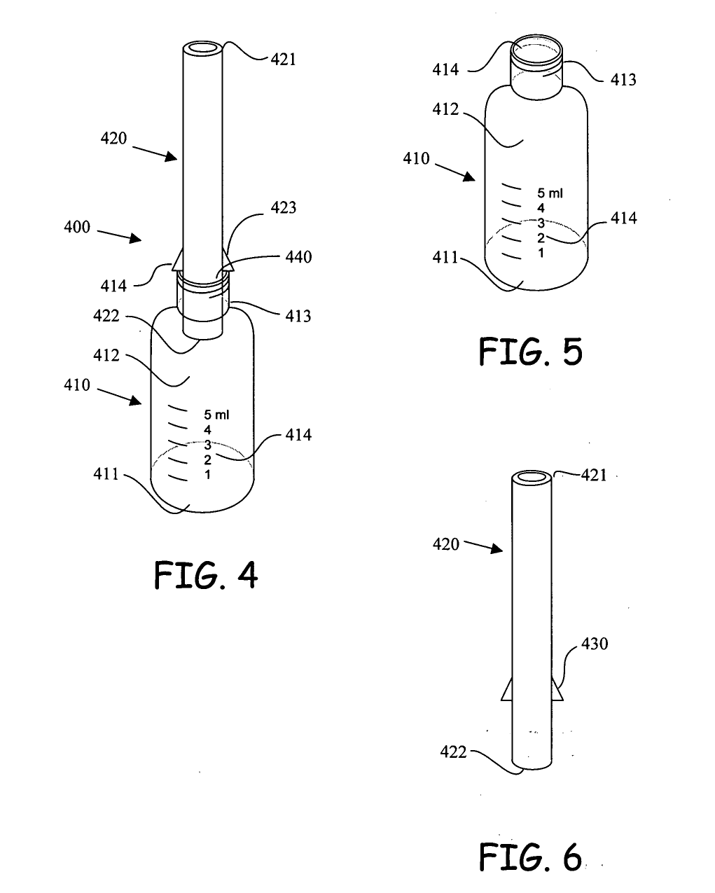

Referring to FIG. 4, in conjunction with FIG. 5, and FIG. 6, in one preferred embodiment of the invention, the oral fluid collection device (400) comprises a specimen collection chamber (410) having a specimen reservoir space (411) and a headspace (412), a vent (440), a tube (420) separated from the collection chamber (410), and a mechanism for connecting the second end of the tube (422) with the headspace (412) of the collection chamber (410) and preventing the tube (420) from being further inserted into the collection chamber (410). The connection mechanism in the particular example device depicted in FIG. 4 includes the neck (413) and the orifice (414) of the collection chamber and the fin-like structure (423) of the tube. The mechanism allows the second end (422) of the tube conveniently inserted into the headspace (412) of the collection chamber and prevents it from being inserted into the specimen reservoir space (411). The fitting structure (423) of the tube (420) at the opening (414) of the collection chamber leaves a vent (440) of the collection chamber. When the tube (420) is connected to the collection chamber (410) through the connecting mechanism of the device (400) an expectorated oral fluid is capable of flowing from the first end (421) of the tube (420) through the second end (422) of the tube (420) and the headspace (412), to the specimen reservoir space (411) and air is capable of escaping the collection chamber through the vent (440).

It is another preferred embodiment of the invention that the oral fluid collection device (100) comprises a volume adequacy system (414). In the particular example device depicted in FIG. 4, the collection chamber is a translucent vial (410) and the volume adequacy system (414) is a volume mark on the wall of the vial (410). When oral fluid is being collected in the collection chamber (410), the collected volume of oral fluid is measured by visually comparing the level of the oral fluid in the specimen reservoir space (411) and the volume mark (414). Other means capable of measuring the volume of the oral fluid specimen in the collection chamber may be adopted as the volume adequacy system of the device, which include a pH paper affixed on the wall of the collection chamber in fluid communication with the specimen reservoir space at certain volume level such that a color change of the pH paper will indicate a volume of the specimen has been reached.

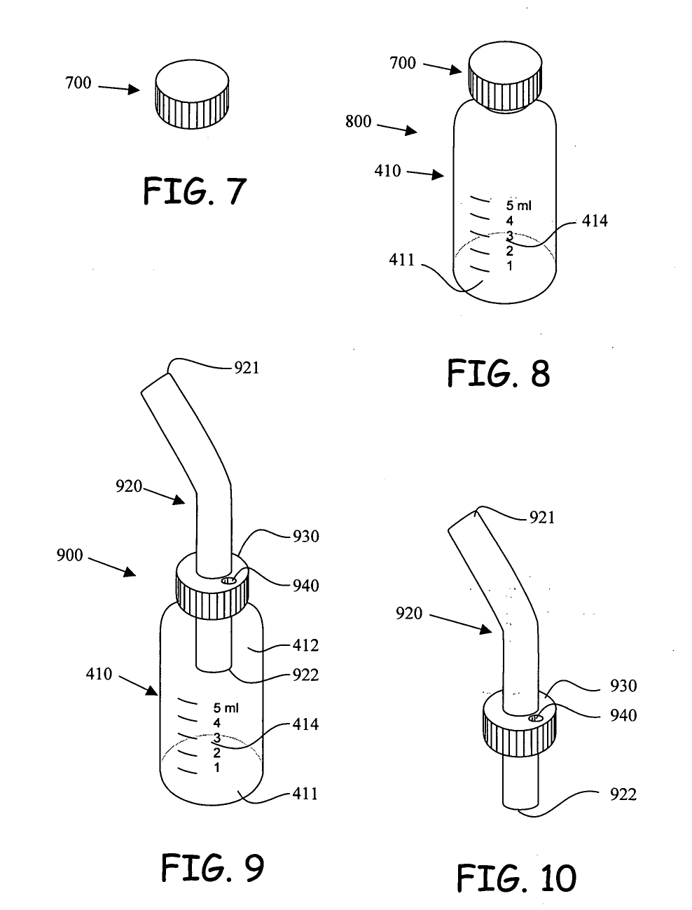

Referring to FIG. 7, in conjunction with FIG. 5 and FIG. 8, the device of a different embodiment of the invention comprises connection mechanism for connecting the specimen collection chamber 410) and the tube of the specimen collection device, which prevents the tube from being removed from the collection chamber 410). The tube (720) of the device (700) comprises a first end (721), a second end (722), and an affixed locking structure (730). With the second end (722) and the locking structure situated inside the collection chamber (410), the locking structure (730) is capable of locking the connection of the tube (720) and the collection chamber (410), such that the second end (722) of the tube (720) is disposed in the headspace (412) of the collection chamber (410) and removing the tube (720) entirely from the collection chamber (410) is prevented by the locking mechanism. When the connection between the collection chamber (410) and the tube (720) is locked by the locking structure (730), a vent (740) of the collection chamber (410) exists, and an expectorated oral fluid is capable of flowing from the first end (721) of the tube (720) through the second end (722) of the tube (720) and the headspace (412), to the specimen reservoir space (411) and air is capable of escaping the collection chamber through the vent (740).

Referring to FIG. 7, in conjunction with FIG. 5, FIG. 12, FIG. 9, FIG. 10, and FIG. 11, in yet another preferred embodiment of the invention the oral fluid collection device (1000) comprises a cap (900) having a collection chamber fitting section (910) and a tube holding section (920). The collection chamber fitting section (910) is capable of mechanically fitting to the neck area (413) of the collection chamber (410) and capping off the collection chamber (410). The tube holding section (920) is capable of removably holding the first end (721) of the tube (700) such that removing the cap (900) will move the tube (720) and the affixed locking structure (730) and locking the connection between the tube (720) and the collection chamber (410). When the tube (720) is locked to the collection chamber (410) through the locking structure (730) and the cap (900) is removed from the first end (721) of the tube (720), an expectorated oral fluid is capable of flowing from the first end (721) of the tube (720) through the second end (722) of the tube (720) and the headspace (412), to the specimen reservoir space (411) and air is capable of escaping the collection chamber through the vent (740). FIG. 10, in conjunction with FIG. 15, depicts the device (700) having optional cap (900) with the components in their shipping positions. One significant benefit of having a cap (900) that is capable of moving the tube (420) is that hand contact with the tube (420) can be avoided.

Referring to FIG. 16, in conjunction with FIG. 5, FIG. 7, FIG. 12, FIG. 17, FIG. 18, FIG. 19, FIG. 20, and FIG. 21, in yet another preferred embodiment of the invention, the oral fluid specimen collection device (1600) comprises a drip cap (1650) and a top cap (1660). The drip cap (1650) comprises a collection chamber fitting section (1651) having a fitting structure (1655) capable of mechanically fitting to the neck area (413) of the collection chamber, a top cap fitting section (1652) having a fitting structure (1653) capable of mechanically fitting to the top cap (1660), a tube holding structure (1656) capable of removably holding the first end (721) of the tube (720) and moving the tube (720) so as to the collection chamber (410), and a through hole (1657). The top cap (1660) comprises a drip cap fitting section (1661) capable of mechanically fitting to the top cap fitting section (1652) of the drip cap and a drip cap sealing section (1662) capable of sealing off the exterior opening (1654) of the drip cap. In an unused device (1600), the tube (720) is preferably held at its first end (721) by the drip cap that is fit to the neck (413) of the collection chamber (4 i 0) and capped off by the top cap (1660). When the drip cap (1650) is removed from such an unused device (1600), the tube (720) inside the collection chamber (410) is moved to the position where it is locked to the collection chamber (410) through the locking structure (730). An expectorated oral fluid is capable of flowing from the first end (721) of the tube (720) through the second end (722) of the tube (720) and the headspace (412), to the specimen reservoir space (411) and air is capable of escaping the collection chamber through the vent (740). Preferably, when pushed with the drip cap (1650), the tube (720) is capable of being unlocked from the connection with the collection chamber (410) and moving back into the collection chamber (410). FIG. 20 depicts the state (2000) of the device (1600) after the tube (720) is pushed back into the collection chamber (410) and capped with both the drip cap (1650) and the top cap (1660). The device (1600) containing the collected oral fluid specimen in its fully capped state (2000) is ready for storage or shipment. FIG. 21 depicts another state (2100) of the device (1600) after specimen collection and the collection chamber (410) is capped with the drip cap (1650) only. In such a state (2100) the oral fluid specimen is easily dripped out of the collection chamber (410), which is better facilitated by squeezing the flexible wall of the collection chamber.

The oral fluid specimen collected using the device of the invention can be tested with an assay method selected from a group including high performance liquid chromatography (HPLC), gas chromatography/mass spectrometry (GC/MS), liquid chromatography (LC), capillary electrophoresis (CE), enzyme linked immunosorbent assay (ELISA), fluorescence immunoassay (FIA), continuous flow fluorescence immunoassay, and immunochromatographic assay, among others, which are familiar to those skilled in the art.

For immediate testing of the collected specimen, an assay reagent capable of reacting with the analyte of the specimen and producing an assay signal can be added into the collection chamber (110). Nevertheless, removing a volume of the collected specimen from the collection chamber (110) to be tested is usually a more preferred way for testing the oral fluid specimen. Means for removing the collected oral fluid specimen from the specimen collection chamber (110) is selected from a group including pouring, dripping, pipetting, vacuuming, and absorbing with an absorbent mass, depending on the specific structure of the device (100) and the type of analysis to be used for analyzing the specimen.

Referring to FIG. 22 and FIG. 23, in another preferred embodiment of the device of the invention, an oral fluid collection and testing device (2200) comprises a collection chamber section (2210) having a specimen reservoir space (2211) and a headspace (2212), a tube (2220) having a first end (2221) and a second end (2212), and an assay reagent section (2250) having an assay reagent component (2260). The second end (2222) of the tube is capable of being connected to the headspace (2212) of the specimen collection section (2210) such that an expectorated oral fluid is capable of flowing from the first end (2221) of the tube (2210) through the second end (2222) of the tube (2210) and the headspace (2212) of the collection section (2210) to the specimen reservoir space (2211) and air is capable of escaping the vent (2240) of the specimen collection section. The assay reagent component (2260) having an assay reagent capable of reacting with an analyte of the oral fluid specimen and producing an assay signal is capable of being in fluid communication with the specimen reservoir space (2211) such that the collected oral fluid specimen is capable of contacting the assay reagent and producing an assay signal. The presence or quantity of the analyte in the oral fluid specimen is determined by detecting the assay signal of the assay reagent section (2250). The assay reagent is selected from a group of reagents capable of reacting with the analyte of the oral fluid specimen and producing an assay signal, which include an enzyme, an enzyme substrate, an antibody, an oxidation or reducing agent, as well as a non-enzymatic signal producing agent, such as colloidal gold, fluorescence label, and color latex. The assay reaction is one of a group consisting enzyme linked immunosorbent assay (ELISA), fluorescence immunoassay (FIA), continuous flow fluorescence immunoassay, enzymatic assay, and reduction-oxidation reaction assay, which are familiar to those skilled in the art. The assay signal is a detectable signal of a group including fluorescence, color change, turbidity change, and electrical property change.

The connection of the assay reagent component (2260) and the specimen reservoir space (2211) varies depending on the nature of the assay reagent component (2260) and the specific design of the device. For example, an orifice between the assay reagent component (2260) and the specimen reservoir space (2211) is capable of allowing a portion of the oral fluid specimen flow from the specimen reservoir space (2211) to the assay reagent component (2260). If the assay reagent component (2260) is in a porous absorbent matrix, an absorbent material is preferably disposed between the specimen reservoir space (2211) and the absorbent assay reagent component (2260), such that the oral fluid specimen of the specimen reservoir space (2211) is capable of flowing from the specimen reservoir space (2211) to the assay absorbent assay reagent component (2260). The device (2200) further comprises an optional cap (2280) capable of capping off the first end (2221) of the tube (2220).

In accordance with a preferred embodiment of the invention, FIG. 22, in conjunction with FIG. 23 in detail depicts a chromatographic immunoassay strip (2260) as the assay reagent component of the device (2200). The specimen collection section (2210) and the assay reagent section (2250) are housed by a plastic housing (2270) formed by an upper piece (2271) and a lower piece (2272). The plastic housing comprises a tube connection port (2230), a specimen collection section (2210) having a specimen reservoir space (2211), a headspace (2212), and a vent (2240), an assay reagent section (2250) having an assay reagent chamber (2251), an orifice (2213) connecting the specimen reservoir space (2211) and the and the assay reagent chamber (2251), and a window (2252) of the assay reagent chamber (2251). The assay reagent chamber (2251) contains chromatographic assay strip (2260), which is a porous substrate comprised of a specimen receiving section (2261), labeled assay reagent section (2262) having a labeled reagent (2263), immobilized assay reagent section (2264) having an immobilized reagent (2265), and a fluid receiving section (2266). The assay principle of chromatographic immunoassay is taught in U.S. Pat. No. 5,591,645 (Rosenstein, 1997) and elsewhere, and is familiar to those skilled in the art. Generally, when a fluid specimen containing an analyte to be tested is exposed the specimen receiving section (2261), the fluid specimen will consecutively flow to the labeled reaction zone (2262), the immobilized reagent section (2264), and the fluid receiving section (2266), and the analyte of the specimen will react with the assay reagents in the respective sections and producing a detectable assay signal at the immobilized reagent section (2264). The result assay signal, preferably a visually readable color change, at the immobilized reagent section is detectable through the window (2252) of the housing (2270). Therefore, when an oral fluid is expectorated to the first end (2221) of the tube (2220) of the oral fluid assay device (2200), the oral fluid will first flow to the specimen reservoir space (2221). A portion of the specimen will flow from the specimen reservoir space (2221) through the orifice (2213) to the specimen receiving section (2261) of the chromatographic assay strip (2260), and continuously flow in a consecutive order through the labeled reagent section (2262), the immobilized reagent section (2264), and the fluid receiving section (2266). Eventually, an assay signal is detected at the window (2262) of the housing (2270) for determining the presence or quantity of the analyte in the oral fluid.

1. A device for collecting an oral fluid specimen for testing the oral fluid comprising
a) a collection chamber having a specimen reservoir space for storing the oral fluid specimen when the specimen is being collected and a head space,
b) a hollow tube having two ends, a first end capable of being inserted into the specimen donor's mouth and a second end capable of being connected to the headspace of the collection chamber,
c) a mechanism for forming a vent of the specimen collection chamber, therefore, an oral fluid expectorated into the first end of the tube is capable of flowing to the specimen reservoir space of the collection chamber while air escapes the collection chamber through the vent.

2. The device of claim 1 wherein the tube and the collection chamber are irremovably connected.

3. The device of claim 1 wherein the tube and the headspace of the collection chamber are separated and are capable of being connected to each other through a connection mechanism.

4. The device of claim 3 wherein the connection mechanism is a structure that allows the second end of the tube to be connected with the headspace of the collection chamber and prevents the tube from being inserted further into the collection chamber.

5. The device of claim 1 wherein the second end of the tube is movably disposed inside the collection chamber and is capable of being locked to the collection chamber in a position that the second end of the tube is away from the specimen reservoir space and is connected to the headspace of the collection chamber.

6. The device of claim 5 wherein the first end of the tube is removably held by a cap of the collection chamber, and, by moving the cape, the tube is capable of being moved to a locked position wherein the second end of the tube is away from the specimen reservoir space and is connected with the headspace of the collection chamber.

7. The device of claim 1 wherein the specimen collection chamber comprises a volume adequacy system capable of indicating the volume adequacy of the collected specimen inside the collection chamber.

8. A method for collecting an oral fluid specimen for testing comprising
a) providing a device comprising
i) a specimen collection chamber having a specimen reservoir space for storing the oral fluid specimen when the specimen is being collected and a headspace,
ii) a hollow tube having two ends, a first end capable of being inserted into the specimen donor's mouth and a second end capable of being connected to the headspace of the collection chamber,
iii) a mechanism for forming a vent of the specimen collection chamber, such that an oral fluid being expectorated into the first end of the tube is capable of flowing to the specimen reservoir area of the collection chamber while air escapes the collection chamber through the vent, and
b) expectorating an oral fluid into the first end of the tube and allowing the oral fluid to flow to the specimen reservoir space of the collection chamber while allowing air to escape the collection chamber through the vent.

9. A device for collecting an oral fluid specimen and detecting an analyte of the oral fluid specimen comprising
a) a specimen collection chamber having a specimen reservoir space for storing the oral fluid specimen when the specimen is being collected and a headspace,
b) a hollow tube having two ends, a first end capable of being inserted into the specimen donor's mouth and a second end capable of being connected to the headspace of the collection chamber,
c) a mechanism for forming a vent of the specimen collection chamber, and
d) an assay reagent component capable of being in fluid communication with the collection chamber,
therefore, an oral fluid being expectorated into the first end of the tube is capable of flowing to the specimen reservoir of the collection chamber and from the collection chamber flowing to the assay reagent component and producing a detectable assay signal.

10. The device of claim 9 wherein the assay reagent component is a chromatographic assay strip.
